(19) United States
(12) Patent Application Publication
(10) Pub. No.: US 2009/0056725 A1
(43) Pub. Date: Mar. 5, 2009

(54) Protective guards and method of using same

(76) Inventor: Farnaz Zand, Burbank, CA (US)

Correspondence Address: JAMES E. BRUNTON, ESQ., P. O. BOX 29000, GLENDALE, CA, 91209, US

(21) Appl. No.: 11/897,278

(22) Filed: Aug. 29, 2007

(51) Int. Cl. A61C 5/14; A61F 11/00

(52) U.S. Cl. 128/859; 128/857

(57) ABSTRACT

A method and apparatus for protecting a user's mouth from discomfort caused by installed dental brackets of the character having a plurality of bracket members attached to the teeth of the user with cooperating wire support members attached to the brackets. The apparatus includes a plurality of uniquely configured protective guards that can be quickly and easily connected to and removed from the bracket members of the installed dental bracket. The protective guards are preferably constructed from a soft, stretchable material and comprise an end guard, a side guard, a corner guard and a front guard, each of which can be readily, removably attached to the dental bracket at selected areas to protect the inside of the user's mouth from injury.

### BACKGROUND OF THE INVENTION

1. Field of the Invention

The present invention relates generally to protective guards designed to protect the user's mouth from damage caused by an installed orthodontic appliance. More particularly, the invention concerns an apparatus and method of using same that comprises a plurality of uniquely configured protective guards that are designed to be removably affixed to the front, side and corner portions of an installed orthodontic appliance to protect the inner lips, mouth and tongue of the user from the wires and brackets of the orthodontic appliance.

2. Discussion of the Prior Art

A common method for aligning misaligned teeth is through the use of dental brackets which typically comprise a plurality of bracket members attached to the teeth of the user with cooperating wire support members attached to the brackets that function to urge the teeth into proper alignment. An undesirable factor of the dental brackets resides in the fact that they have a tendency to cause significant discomfort to the user and sometimes injury to the user's mouth due to the engagement of the wires and the bracket members with the inside of the user's mouth.

A number of attempts have been made in the past to solve this troublesome problem by providing various types of pads, shields and other apparatus that can be removably affixed to the dental brace by adhesives and the like. However, these attempts have been generally unsuccessful for various reasons, including the complexity of the devices, the fact that the devices are cumbersome and difficult to use and the inability of the devices to be effectively secured in place. As will become apparent from the discussion that follows, none of the prior art devices offer the comfort and relative ease of use as do the devices of the present invention.

### SUMMARY OF THE INVENTION

It is an object of the present invention to provide a method and apparatus for protecting a user's mouth from discomfort caused by installed dental brackets of the character having a plurality of bracket members attached to the teeth of the user with cooperating wire support members attached to the brackets.

Another object of the invention is to provide an apparatus of the aforementioned character that includes a plurality of uniquely configured protective guards that can be quickly and easily connected to and removed from the bracket members of the installed dental bracket.

Another object of the invention to provide an apparatus of the character described in the preceding paragraphs in which the plurality of protective guards are constructed from a soft, stretchable material and comprise an end guard, a side guard, a corner guard and a front guard, each of which can be readily, removably attached to the dental bracket at selected areas to protect the inside of the user's mouth from injury.

Another object of the invention is to provide protective guards of the character described that are positioned over the dental bracket by simply placing the various guard components of the apparatus over the irritating areas of the dental bracket and then anchoring the end portions of the protective guards with the spaced-apart bracket members provided on the dental bracket.

Another object of the invention is to provide an apparatus as described in the preceding paragraphs that comprises protective guards that are easy to use, provides maximum comfort, remain securely in place, provide immediate relief and can be reused if desired.

The foregoing objectives, as well as other objectives of the invention, will be achieved by the method and apparatus of the invention, the details of which will be described hereinafter.

### BRIEF DESCRIPTION OF THE DRAWINGS

FIG. 1 is a generally perspective view of a portion of the inside of a user's mouth illustrating a pair of orthodontic braces affixed to the teeth of the user and showing one form of the front protective guard of the plurality of protective guards of the invention affixed to a pair of spaced-apart bracket members provided on one of the orthodontic braces.

FIG. 2 is a front view of the front protective guard of the invention shown in FIG. 1.

FIG. 3 is a generally perspective view of another portion of the user's mouth and showing one form of the end protective guard component of the invention in position over the installed orthodontic brace.

FIG. 4 is a front view of the end protective guard of the invention shown in FIG. 3.

FIG. 5 is a generally perspective view of another portion of the user's mouth showing one form of the side protective guard component of the invention in position over the installed orthodontic brace.

FIG. 6 is a front view of the side protective guard of the invention shown in FIG. 5.

FIG. 7 is a generally perspective view of another portion of the user's mouth and showing the corner protective guard component of the invention in position over the installed orthodontic brace.

FIG. 8 is a front view of the corner protective card of the invention shown in FIG. 7.

### DESCRIPTION OF THE INVENTION

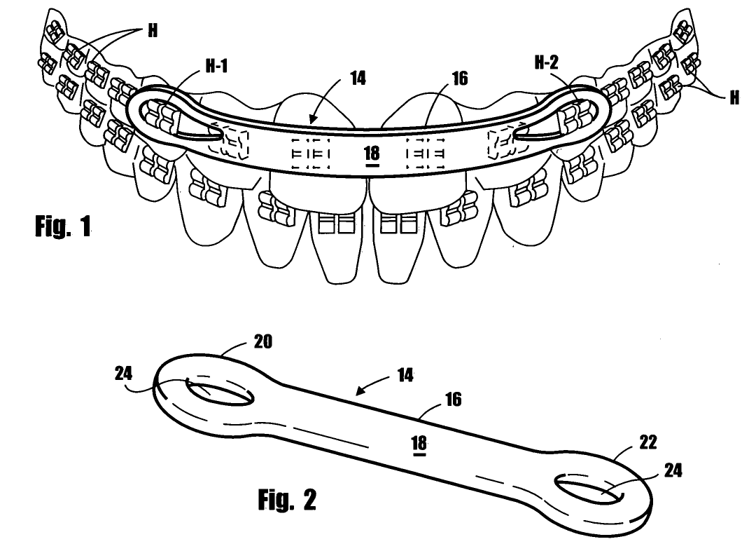

Referring to the drawings and particularly to FIGS. 1 and 2, one form of the protective guard of the plurality of protective guards of the apparatus of the invention is there shown affixed to one of a pair of orthodontic braces “B” shown as they appear when affixed to the teeth of the user in a conventional manner. The orthodontic brackets “B” typically comprise a plurality of spaced-apart bracket hooks “H” that are attached to the teeth of the user and a cooperating wire “W” that function to urge the teeth into proper alignment. While the protective guards the invention can be constructed of various materials, for example, material such as soft latex rubber and silicone have proven satisfactory.

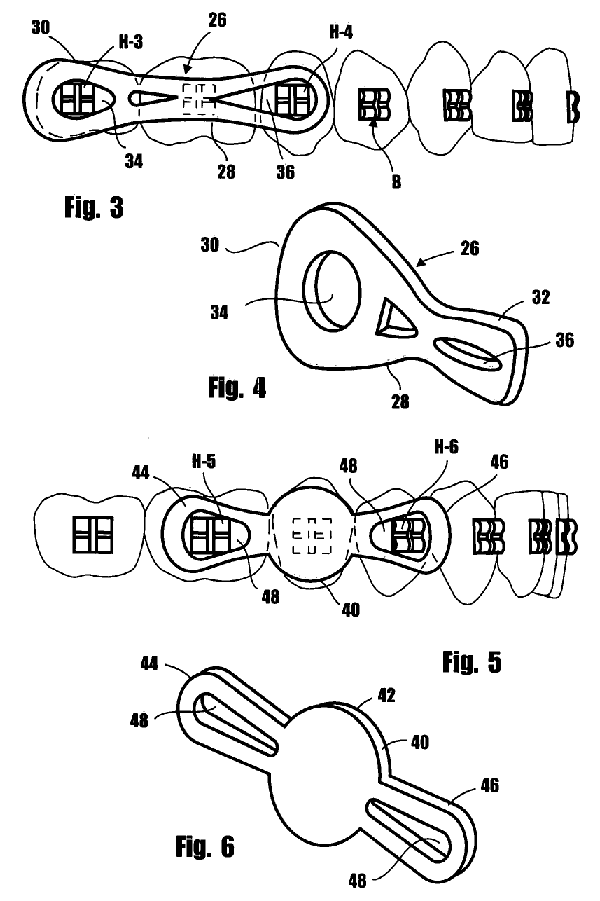

The protective guard of the invention shown in FIGS. 1 and 2 comprises the front protective guard 14 of a plurality of strategically configured protective guards of the character illustrated in FIGS. 3 through 8 of the drawings. As best seen in FIG. 2, front protective guard 14, which is preferably constructed from a generally stretchable, formable material, comprises a body 16 having an elongated central portion 18 and a pair of spaced-apart end portions 20 and 22. Each of the end portions 20 and 22 have an aperture 24 for receiving a selected one of the spaced-apart bracket hooks “H” of the orthodontic appliance in the manner illustrated in FIG. 1 of the drawings. As indicated in FIG. 1, the body portion 18 of protective guard 14, which is of a generally uniform thickness, has a length “L”, a width “W” which is substantially less than the length “L”.

In accordance with one form of the method of the invention, front protective guard 14 can be installed over the front portion of the selected one of the orthodontic appliances by placing one of the openings 24 over a first bracket hook “H-1”, stretching the protective guard slightly and then inserting the other of the openings 24 over a second bracket hook “H-2” that is spaced-apart from the first bracket hook (see FIG. 1). Due to the strategic configuration of front protective guard 14, once positioned over the orthodontic appliance in the manner described, it will remain securely in position until removed by the user.

Referring next to FIG. 3, which is a generally perspective view of another portion of the user's mouth, one form of the end protective guard component 26 of the invention is there shown in position over an installed orthodontic brace “B”. As best seen in FIG. 4 of the drawings, the end protective guard component 26, which is another of the plurality of protective guards of the apparatus of the invention, comprises a central portion 28 and generally oval-shaped first and second end portions 30 and 32. First end portion 30 has a generally circular-shaped aperture 34, while a second end portion 32 has a generally elliptical-shaped aperture 36. As before, each of the apertures 34 and 36 is adapted to closely receive a selected one of a pair of spaced-apart bracket hooks “H-3” and “H-4” of the orthodontic appliance in the manner shown in FIG. 3 of the drawings. More particularly, the end protective guard 26 can be installed over the end portion of the selected one of the orthodontic appliances by placing opening 34 over bracket hook “H-3”, stretching the protective guard slightly and then inserting the other of the openings, 36, over bracket hook “H-4” (see FIG. 3). Due to the strategic configuration of end protective guard 26, once positioned over the orthodontic appliance in the manner described, it will remain securely in position until removed by the user.

Turning to FIGS. 5 and 6, another form of the protective guard component of the apparatus of the invention is there shown and generally designated by the numeral 40. As best seen in FIG. 6 of the drawings, the side protective guard component 40, comprises a generally circular-shaped central portion 42 and generally oval-shaped first and second end portions 44 and 46, each of which is provided with a generally elliptical-shaped apertures 48. As before, each of the apertures 48 is adapted to closely receive a selected one of a pair of spaced-apart bracket hooks “H-5” and “H-6” of the orthodontic appliance in the manner shown in FIG. 5 of the drawings. More particularly, the side protective guard 40 can be installed over the side portion of the selected one of the orthodontic appliances by placing opening 48 over bracket hook “H-5”, stretching the protective guard slightly and then inserting the other of the openings 48 over bracket hook “H-6” (see FIG. 5). Due to the strategic configuration of side protective guard 40, once positioned over the orthodontic appliance in the manner described, it will remain securely in position until removed by the user.

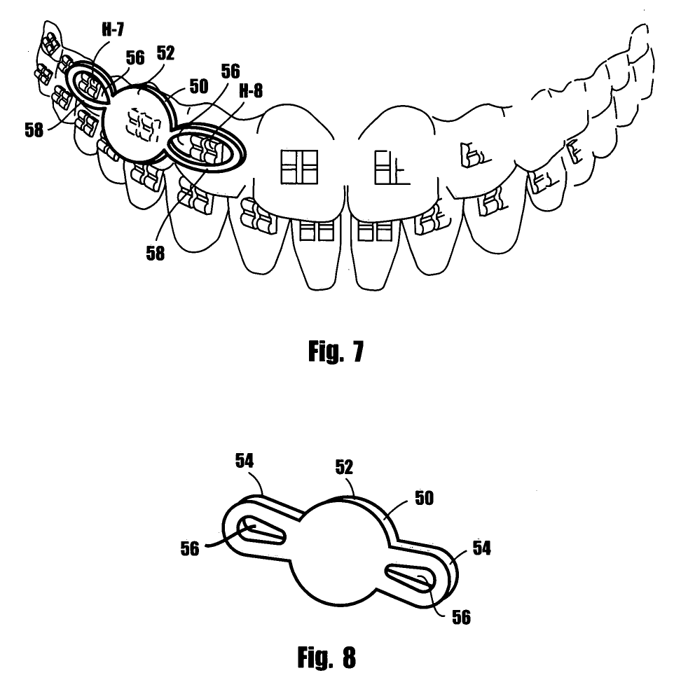

Referring next to FIGS. 7 and 8, yet another form of the protective guard component of the apparatus of the invention is there shown and generally designated by the numeral 50. This component, which comprises the corner guard component of the apparatus, is somewhat similar in construction to the side guard component 40 and includes a generally circular-shaped central portion 52 and a pair of spaced-apart end portions 54, each having a generally elliptical-shaped aperture 56. As illustrated in FIG. 7 of the drawings, each of the apertures 56 is adapted to closely receive a selected one of a pair of spaced-apart bracket hooks “H-7” and “H-8” of the orthodontic appliance in the manner shown in FIG. 5 of the drawings. More particularly, the corner protective guard 50 can be installed over a corner portion of the selected one of the orthodontic appliances by placing one of the openings 56 over bracket hook “H-7”, stretching the protective guard slightly and then inserting the other of the openings 56 over bracket hook “H-8” (see FIG. 7). Due to the strategic configuration of corner protective guard 50, once positioned over the orthodontic appliance in the manner described, it will remain securely in position until removed by the user.

Considering now the methods of the invention for protecting the user's mouth from injury caused by an installed orthodontic appliance having a central portion, a side portion and a plurality of spaced-apart bracket hooks. One form of the method of the invention involves the use of a first stretchable, protective guard, such as guard 14 having an elongated central portion 18 and a pair of spaced-apart end portions 24. This form of the method of the invention concerns emplacement of the protective guard 14 over the central portion of the orthodontic appliance in a manner to protect the inner lips and mouth of the user. Placement of the protective guard 14 over the central portion of the orthodontic appliance is accomplished by first placing a selected one of the hook-receiving openings of the protective guard over a selected bracket hook located proximate the central portion of the appliance. Next, the protective guard is stretched longitudinally so that the other opening in the protective guard can be placed over a second, spaced-apart bracket hook located proximate the central portion of the appliance.

Another form of the method of the invention involves the use of a second stretchable, protective guard, such as end guard 26 having a central portion 28 and a pair of spaced-apart end portions 30 and 32. This form of the method of the invention concerns emplacement of the protective guard 26 over the end portion of the orthodontic appliance in a manner to protect the sides of the mouth of the user. Placement of the protective guard 26 over the end portion of the orthodontic appliance is accomplished by first placing opening 34 of the protective guard over a selected bracket hook located proximate one side portion of the appliance. Next, the protective guard is stretched longitudinally so that the other opening 36 in the protective guard can be placed over a second, spaced-apart bracket hook located proximate the end portion of the appliance.

Still another form of the method of the invention involves the use of a third stretchable, protective guard, such as guard 40 having a generally circular-shaped central portion 42 and a pair of spaced-apart end portions 44 and 46. This form of the method of the invention concerns emplacement of the protective guard 40 over the side portion of the orthodontic appliance in a manner to protect the sides of the mouth of the user. Placement of the protective guard 40 over the side portion of the orthodontic appliance is accomplished by first placing opening 48 of the protective guard over a selected bracket hook located proximate one side portion of the appliance. Next, the protective guard is stretched longitudinally so that the other opening 48 in the protective guard can be placed over a second, spaced-apart bracket hook located proximate the central portion of the appliance.

Due to the unique configuration of the protective guards in the materials from which they are fabricated, once the protective guards are interconnected with the orthodontic appliance in the manner described in the preceding paragraphs, they remain securely in place until the user desires to remove them. Because of the strategic positioning of the protective guards, experience has shown that their use substantially reduces the discomfort caused to the user by the installed orthodontic appliance.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A protective guard for protecting the user's mouth from irritation caused by an installed orthodontic appliance having a plurality of bracket hooks, said protective guard comprising an elongated strip of generally stretchable, formable material having a body portion and plurality of spaced-apart openings formed in said body portion for receiving selected spaced-apart bracket hooks for positioning said protective guard over a selected portion of the orthodontic appliance.

2. The protective guard as defined in claim 1 in which said body portion of said protective guard comprises an elongated central portion and a pair of spaced-apart end portions, each of said end portions having an aperture for receiving a selected one of a pair of spaced-apart bracket hooks of the orthodontic appliance.

3. The protective guard as defined in claim 1 in which said body portion of said protective guard comprises a generally circular-shaped central portion and a pair of spaced-apart end portions, each of said end portions having an aperture for receiving a selected one of a pair of spaced-apart bracket hooks of the orthodontic appliance.

4. The protective guard as defined in claim 1 in which said body portion of said protective guard comprises generally oval-shaped first and second end portion, each said first and second end portions having an aperture for receiving a selected one of a pair of spaced-apart bracket hooks of the orthodontic appliance.

5. The protective guard as defined in claim 1 in which said body portion is constructed from silicone.

6. The protective guard as defined in claim 1 in which said body portion is constructed from latex rubber.

7. The protective guard as defined in claim 1 in which said body portion has a length, a width and a generally uniform thickness, said width being greater than said length.

8. An apparatus for protecting the user's mouth from injury caused by an installed orthodontic appliance having a front portion, a side portion, end portions, a corner portion and a plurality of spaced-apart bracket hooks, said apparatus comprising:
(a) a front guard for covering at least a part of the front portion of the orthodontic appliance, said front guard comprising an elongated strip of generally stretchable, formable material having a body portion and plurality of spaced-apart openings formed in said body portion for receiving selected spaced-apart bracket hooks for positioning said protective guard over the central portion of the orthodontic appliance; and
(b) a side guard for covering at least a part of the side portion of the orthodontic appliance, said side guard comprising a strip of generally stretchable, formable material having a body portion, a first end portion having an opening formed therein for receiving a selected one of the spaced-apart bracket hooks and a second end portion having an opening formed therein for receiving a selected one of the spaced-apart bracket hooks.

9. The apparatus as defined in claim 8 further including an end guard for covering at least a part of the end portion of the orthodontic appliance, said end guard comprising a strip of generally stretchable, formable material having a body portion and a plurality of spaced-apart openings formed in said body portion for receiving selected spaced-apart bracket hooks for positioning said protective guard over at least a part of the end portion of the orthodontic appliance.

10. The apparatus as defined in claim 8 further comprising a corner guard for covering the corner portion of the orthodontic appliance, said corner guard comprising a strip of generally stretchable, formable material having a generally circular-shaped body portion, a first end portion having an opening formed therein for receiving a selected one of the spaced-apart bracket hooks and a second end portion having an opening formed therein for receiving a selected one of the spaced-apart bracket hooks.

11. A method of protecting the user's mouth from injury caused by an installed orthodontic appliance having a plurality of spaced-apart bracket hooks through the use of a stretchable, protective guard having a central portion and a pair of spaced-apart end portions, each said end portion having a hook-receiving aperture, said method comprising the steps of:
(a) placing a selected one of the hook-receiving openings of the protective guard over a selected bracket hook;
(b) stretching the protective guard; and
(c) inserting the other of the hook-receiving openings of the protective guard over a second, spaced-apart bracket hook.

12. A method of protecting the user's mouth from injury caused by an installed orthodontic appliance having a central portion, a side portion and a plurality of spaced-apart bracket hooks through the use of a first stretchable, protective guard having an elongated central portion and a pair of spaced-apart end portions, each said end portion having a hook-receiving aperture and through the use of a second stretchable, protective guard having a generally circular-shaped central portion and a pair of spaced-apart end portions, each said end portion having a hook-receiving aperture, said method comprising the steps of:
(a) placing a selected one of the hook-receiving openings of the first protective guard over a selected bracket hook located proximate the central portion of the appliance;
(b) stretching the first protective guard;
(c) inserting the other of the hook-receiving openings of the first protective guard over a second, spaced-apart bracket hook located proximate the central portion of the appliance;
(d) placing a selected one of the hook-receiving openings of the second protective guard over a selected bracket hook located proximate the side portion of the appliance;
(e) stretching the second protective guard; and
(f) inserting the other of the hook-receiving openings of the second protective guard over a second, spaced-apart bracket hook located proximate the side portion of the appliance.
